(19) United States
(12) Patent Application Publication
(10) Pub. No.: US 2012/0100188 A1
(43) Pub. Date: Apr. 26, 2012

(54) SOLID STATE FORMS OF PALIPERIDONE SALTS AND PROCESS FOR THE PREPARATION THEREOF

(75) Inventors: Girish Dixit, Uttar Pradesh (IN); Anil Shahaji Khile, Maharashtra (IN); Vignesh Nair, Kerala (IN); Nitin Sharadechandra Pradhan, Maharashtra (IN)

(73) Assignee: Actavis Group PTC ehf, Hafnarfjordur (IS)

(21) Appl. No.: 13/318,664

(22) PCT Filed: May 25, 2010
(86) PCT No.: PCT/IB2010/001375

§ 371 Date: Dec. 30, 2011

(30) Foreign Application Priority Data
May 28, 2009 (IN) 1232/CHE/2009

(51) Int. Cl. A61K 31/519; A61K 9/14; C07D 471/04

(52) U.S. Cl. 424/400; 544/282; 514/259.41

(57) ABSTRACT

Provided herein are solid state forms of paliperidone salts, processes for preparation, pharmaceutical compositions, and method of treating thereof. Paliperidone is represented by the following structural formula (I): More particularly, provided are solid state forms of paliperidone acid addition salts, wherein the acid counter ion is provided by an acid selected from the group consisting of L-(+)-tartaric acid, p-toluenesulfonic acid, maleic acid, oxalic acid, fumaric acid, acetic acid and malic acid. Provided also herein is a process for preparing substantially pure paliperidone free base using the solid state forms of paliperidone salts.

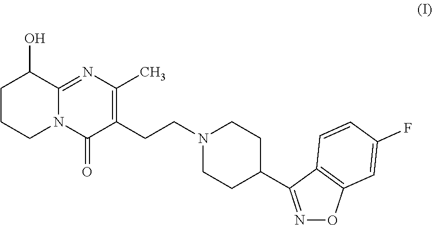

### CROSS REFERENCE TO RELATED APPLICATION

This application claims the benefit of priority to Indian provisional application No. 1232/CHE/2009, filed on May 28, 2009, which is incorporated herein by reference in its entirety.

### FIELD OF THE INVENTION

The present invention relates to novel solid state forms of paliperidone salts, process for their preparation, pharmaceutical compositions, and method of treating thereof.

### BACKGROUND

U.S. Pat. Nos. 4,804,663 and 5,158,952 disclose a variety of 3-piperidinyl-1,2-benzisoxazole derivatives, processes for their preparation, pharmaceutical compositions comprising the derivatives, and methods of use thereof. These compounds have long-acting antipsychotic properties and are useful in the treatment of warm-blooded animals suffering from psychotic diseases. Among them, paliperidone, (±)-3-[2-[4-(6-fluoro-1,2-benzisoxazol-3-yl)-1-piperidinyl]ethyl]-6,7,8,9-tetrahydro-9-hydroxy-2-methyl-4H-pyrido[1,2-a]pyrimidin-4-one, is an antipsychotic agent and is indicated for the both acute (short-term) and maintenance (long-term) treatment of schizophrenia. Paliperidone is represented by the following structural formula:

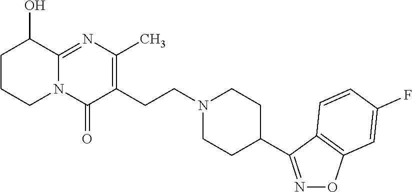

Paliperidone (available as INVEGA®) is an atypical antipsychotic developed by Janssen Pharmaceutica.

Processes for the preparation of paliperidone and related compounds are disclosed in U.S. Pat. Nos. 5,158,952; 5,254,556; 5,688,799 and 6,320,048.

According to U.S. Pat. No. 5,158,952 (hereinafter referred to as the '952 patent), paliperidone is prepared by the reaction of 3-(2-chloroethyl)-6,7,8,9-tetrahydro-9-hydroxy-2-methyl-4H-pyrido[1,2-a]-pyrimidin-4-one with 6-fluoro-3-(4-piperidinyl)-1,2-benzisoxazole in the presence of a base in a reaction inert solvent and optionally in the presence of a phase transfer catalyst. The reaction mixture containing paliperidone obtained is then subjected to evaporation, and the oily residue is extracted with trichloromethane followed by water washings. The organic layer is dried, filtered and evaporated, followed by column chromatographic purifications over silica gel using a mixture of trichloromethane and methanol. The pure fractions are collected and the eluent is evaporated. The resulting residue is crystallized from 2-propanone. After cooling, the precipitated product is filtered off, washed with a mixture of 2-propanol and 2,2′-oxybispropane, and recrystallized from 2-propanol to produce paliperidone.

While the '952 patent mentions that some of the disclosed compounds can form salts with acids, for example, inorganic acids such as hydrochloric acid, hydrobromic acid and the like, sulfuric acid, nitric acid, phosphoric acid and the like; or organic acids, such as, for example, acetic, propanoic, hydroxyacetic, 2-hydroxypropanoic, 2-oxopropanoic, ethanedioic, propanedioic, butanedioic, (Z)-2-butenedioic, (E)-2-butenedioic, 2-hydroxybutanedioic, 2,3-dihydroxybutanedioic, 2-hydroxy-1,2,3-propanetricarboxylic, methanesulfonic, ethanesulfonic, benzenesulfonic, 4-methylbenzenesulfonic, cyclohexanesulfamic, 2-hydroxybenzoic, 4-amino-2-hydroxybenzoic and the like acids; no salts of the disclosed compounds had been prepared or isolated.

PCT Publication No. WO2009016653 discloses a process for the preparation of paliperidone hydrochloride salt.

U.S. Application No. 20090036470 discloses a process for the preparation of crystalline paliperidone hydrochloride salt.

PCT Publication No. WO2009060297 (hereinafter referred to as the '297 application) describes certain acid addition salts of paliperidone derived from an acid selected from hydrochloric acid, hydrobromic acid, hydroiodic acid, ortho phosphoric acid, fumaric acid or oxalic acid. The '297 publication further discloses crystalline forms of paliperidone hydrochloride, paliperidone hydrobromide, paliperidone phosphate and paliperidone fumarate, and characterizes them by powder X-ray diffraction. According to the '297 application, the paliperidone fumarate, which we denote as crystalline Form I, is characterized by an XRD pattern (2-theta) (±0.2 degrees) having characteristics peaks at 7.60, 10.73, 13.81, 15.23, 17.34, 19.08, 20.27, 22.94, 24.16, 25.83 and 27.64 degrees with further peaks at 8.21, 10.32, 11.44, 14.65, 15.89, 18.52, 21.04, 24.75, 26.98 and 29.11 degrees.

There remains a need for novel solid state forms of paliperidone acid addition salts.

### SUMMARY OF THE INVENTION

In one aspect, provided herein are novel solid state forms of a paliperidone salt, wherein the salt is a tartrate salt, a tosylate salt, a maleate salt, an oxalate salt, an acetate salt, or a malate salt.

In another aspect, paliperidone salts in a crystalline form are provided. In yet another aspect, paliperidone salts in an amorphous form are provided. In still another aspect, the solid state forms of paliperidone salts exist in an anhydrous and/or solvent-free form or as a hydrate and/or a solvate form.

In another aspect, encompassed herein is a process for preparing a solid state form of a paliperidone salt comprising contacting paliperidone free base with an acid in a suitable solvent under suitable conditions to produce a reaction mass, and isolating the solid state form of paliperidone acid addition salt, wherein the acid addition salt of paliperidone is a tartrate salt, a tosylate salt, a maleate salt, an oxalate salt, an acetate salt or a malate salt.

In another aspect, provided herein is a novel and stable crystalline form of paliperidone fumarate, designated herein as paliperidone fumarate crystalline Form II, characterized by an X-ray powder diffraction pattern having peaks expressed as 2-theta angle positions at about 9.93, 10.65, 11.43, 14.16, 15.79, 17.43, 18.99, 20.17, 20.58, 21.45, 24.09 and 25.70±0.2 degrees.

The crystalline Form II of paliperidone fumarate is differentiated from the crystalline Form I, disclosed in the prior art, by an X-ray powder diffraction pattern in absence of peaks expressed as 2-theta angle positions at about 7.60, 8.21, 13.81, 15.23, 26.98 and 29.11±0.2 degrees.

In another aspect, encompassed herein is a process for preparing the substantially pure and stable crystalline Form II of paliperidone fumarate.

In another aspect, provided herein is a method for treating a patient suffering from psychotic diseases; comprising administering a solid state form of paliperidone salt, or a pharmaceutical composition that comprises the solid state form of paliperidone salt along with pharmaceutically acceptable excipients, wherein the salt of paliperidone is a tartrate salt, a tosylate salt, a maleate salt, an oxalate salt, an acetate salt or a malate salt.

In another aspect, encompassed herein is a process for preparing highly pure paliperidone free base by using the solid state forms of paliperidone salts disclosed herein.

In another aspect, provided herein is a pharmaceutical composition that comprises a solid state forms of a paliperidone salt as disclosed herein, and one or more pharmaceutically acceptable excipients.

In still another aspect, provided herein is a pharmaceutical composition that comprises a solid state form of a paliperidone salt made by the process disclosed herein, and one or more pharmaceutically acceptable excipients.

In still further aspect, encompassed is a process for preparing a pharmaceutical formulation comprising combining any one of the solid state forms of paliperidone salts disclosed herein with one or more pharmaceutically acceptable excipients.

In another aspect, the solid state forms of paliperidone salts disclosed herein for use in the pharmaceutical compositions have a D₉₀ particle size of less than or equal to about 500 microns, specifically about 1 micron to about 300 microns, and most specifically about 10 microns to about 150 microns.

### BRIEF DESCRIPTION OF THE DRAWINGS

FIG. 1 is a characteristic powder X-ray diffraction (XRD) pattern of crystalline paliperidone L-(+)-tartrate salt.

FIG. 2 is a characteristic differential scanning calorimetric (DSC) thermogram of crystalline paliperidone L-(+)-tartrate salt.

FIG. 3 is a characteristic powder X-ray diffraction (XRD) pattern of crystalline paliperidone tosylate.

FIG. 4 is a characteristic differential scanning calorimetric (DSC) thermogram of crystalline paliperidone tosylate.

FIG. 5 is a characteristic powder X-ray diffraction (XRD) pattern of crystalline paliperidone maleate.

FIG. 6 is a characteristic differential scanning calorimetric (DSC) thermogram of crystalline paliperidone maleate.

FIG. 7 is a characteristic powder X-ray diffraction (XRD) pattern of crystalline paliperidone oxalate.

FIG. 8 is a characteristic differential scanning calorimetric (DSC) thermogram of crystalline paliperidone oxalate.

FIG. 9 is a characteristic powder X-ray diffraction (XRD) pattern of crystalline Form II of paliperidone fumarate.

FIG. 10 is a characteristic differential scanning calorimetric (DSC) thermogram of crystalline Form II of paliperidone fumarate.

FIG. 11 is a characteristic powder X-ray diffraction (XRD) pattern of crystalline paliperidone acetate.

FIG. 12 is a characteristic differential scanning calorimetric (DSC) thermogram of crystalline paliperidone acetate.

FIG. 13 is a characteristic powder X-ray diffraction (XRD) pattern of crystalline paliperidone malate.

FIG. 14 is a characteristic differential scanning calorimetric (DSC) thermogram of crystalline paliperidone malate.

### DETAILED DESCRIPTION OF THE INVENTION

Solid state forms of paliperidone salts, except hydrochloride, hydrobromide, phosphate and fumarate salts, have not been reported, isolated, or characterized in the literature. The present inventors have surprisingly and unexpectedly found that some of the acid addition salts of (±)-3-[2-[4-(6-fluoro-1,2-benzisoxazol-3-yl)-1-piperidinyl]ethyl]-6,7,8,9-tetrahydro-9-hydroxy-2-methyl-4H-pyrido[1,2-a]pyrimidin-4-one, i.e., paliperidone salts, specifically, tartrate, tosylate, maleate, oxalate, acetate and malate salts, can be isolated as solid state forms.

It has also been found that the solid state forms of paliperidone salts are useful intermediates in the preparation of paliperidone or a pharmaceutically acceptable salt thereof in high purity. The solid state forms of paliperidone salts have good flow properties and are stable at room temperature, enhanced temperature, at relative high humidities, and in aqueous media. The novel solid state forms of paliperidone salts are suitable for formulating paliperidone.

In the formulation of drug compositions, it is important for the active pharmaceutical ingredient to be in a form in which it can be conveniently handled and processed. Convenient handling is important not only from the perspective of obtaining a commercially viable manufacturing process, but also from the perspective of subsequent manufacture of pharmaceutical formulations (e.g., oral dosage forms such as tablets) comprising the active pharmaceutical ingredient.

Chemical stability, solid state stability, and “shelf life” of the active pharmaceutical ingredient are important properties for a pharmaceutically active compound. The active pharmaceutical ingredient, and compositions containing it, should be capable of being effectively stored over appreciable periods of time, without exhibiting a significant change in the physico-chemical characteristics of the active pharmaceutical ingredient, e.g., its chemical composition, density, hygroscopicity and solubility. Thus, in the manufacture of commercially viable and pharmaceutically acceptable drug compositions, it is important, wherever possible, to provide the active pharmaceutical ingredient in a stable form.

New solid state forms of a pharmaceutical agent can further the development of formulations for the treatment of illnesses. For instance, solid forms of a compound are known in the pharmaceutical arts to affect, for example, the solubility, dissolution rate, bioavailability, chemical and physical stability, flowability, fractability, and compressibility of the compound, as well as the safety and efficacy of drug products based on the compound.

The discovery of novel salts in solid state forms of pharmaceutically useful compounds provides a new opportunity to improve the performance characteristics of a pharmaceutical product. It also adds value to the material that a formulation scientist can use the same for designing, for example, a pharmaceutical dosage form of a drug with a targeted release profile or other desired characteristic.

According to another aspect, provided herein are novel and stable solid state forms of paliperidone salts, wherein the salt of paliperidone is an L-(+)-tartrate salt, a tosylate salt, a maleate salt, an oxalate salt, an acetate salt or a malate salt.

It has been surprisingly and unexpectedly found that some of the solid state forms of paliperidone salts disclosed herein have better solubility and dissolution properties. A comparative data related to the solubility properties of paliperidone and the solid state forms of paliperidone salts is furnished in the Example 9 as disclosed herein.

In one embodiment, the solid state forms of paliperidone salts exist in a crystalline form. In another embodiment, the solid state forms of paliperidone salts exist in an amorphous form. In another embodiment, the solid state forms of paliperidone salts exist in an anhydrous and/or solvent-free form, or as a hydrate and/or a solvate form. Such solvated or hydrated forms may be present as hemi-, mono-, sesqui-, di- or tri-solvates or hydrates. Solvates and hydrates may be formed as a result of solvents used during the formation of the paliperidone salts becoming embedded in the solid lattice structure. Because formation of the solvates and hydrates occurs during the preparation of paliperidone salts, formation of a particular solvated or hydrated form depends greatly on the conditions and method used to prepare the salt. Solvents should be pharmaceutically acceptable.

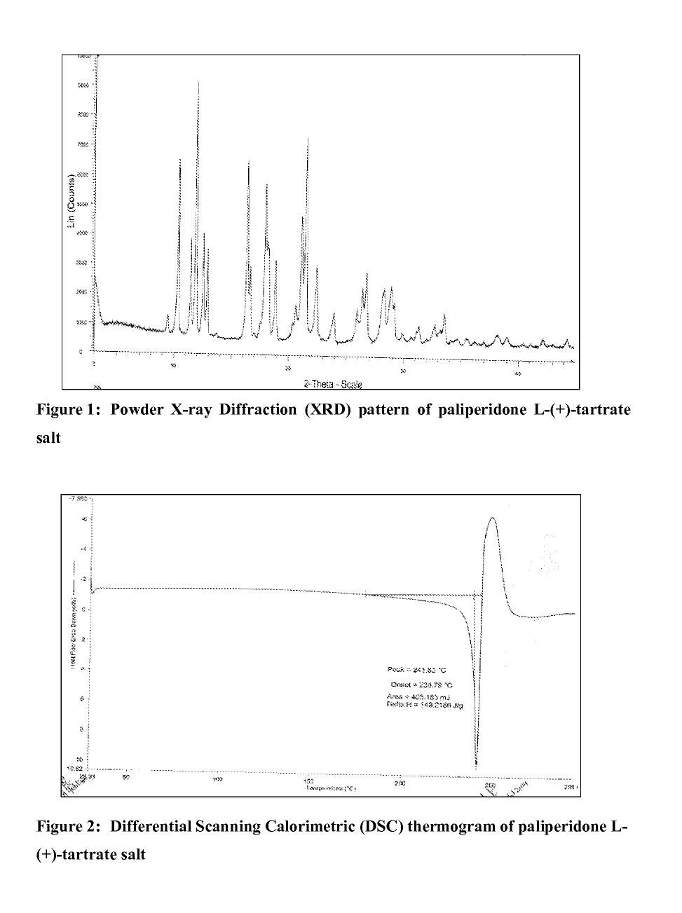

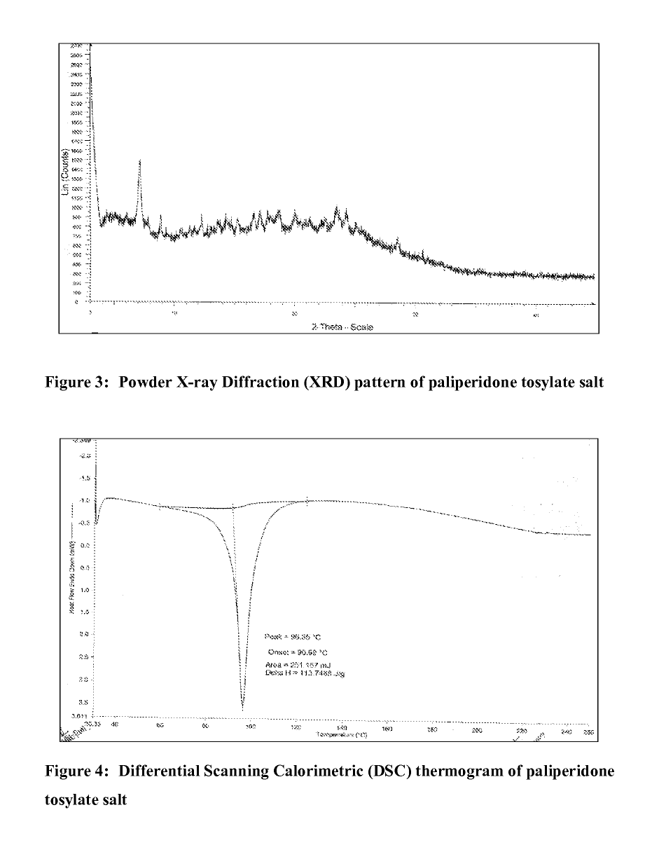

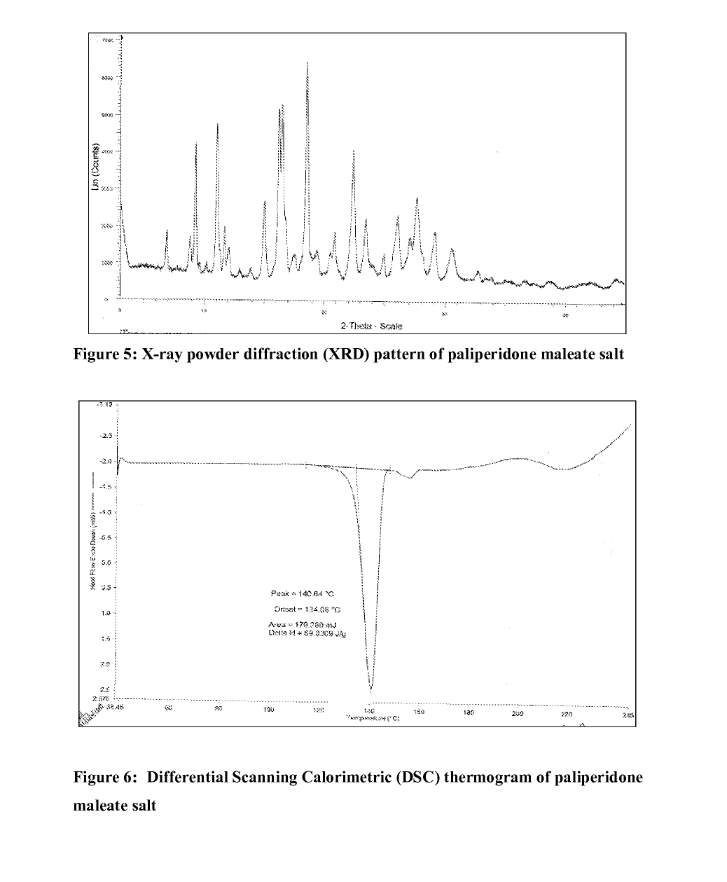

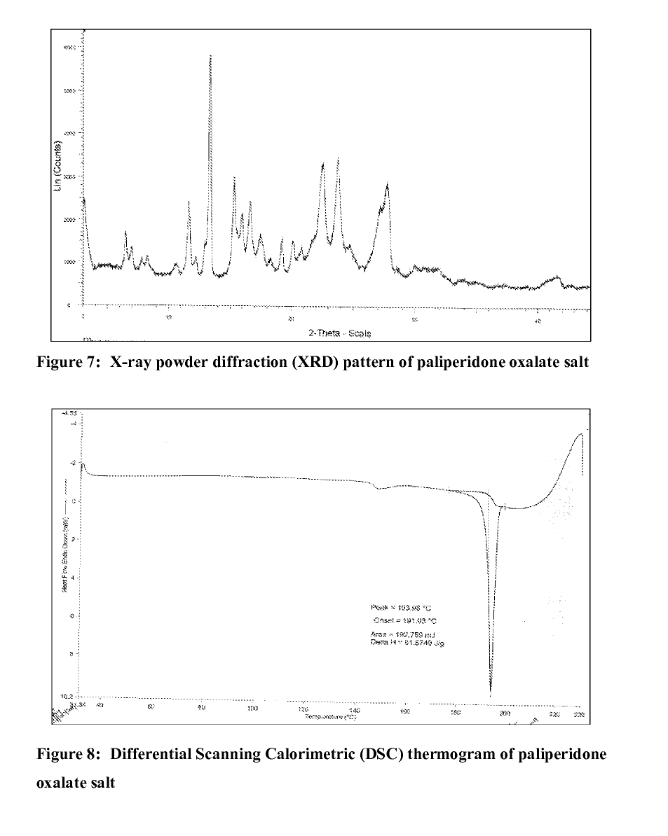

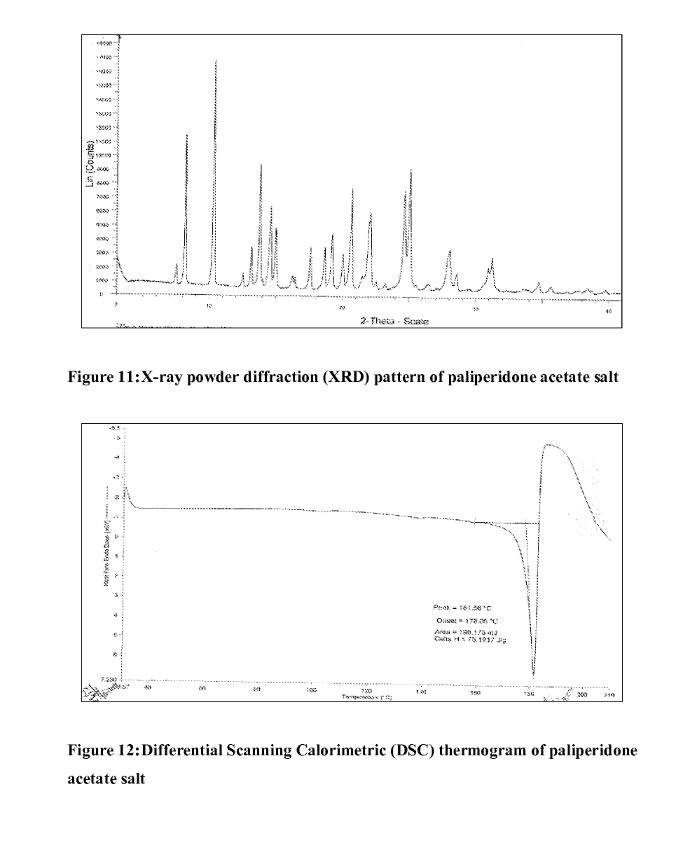

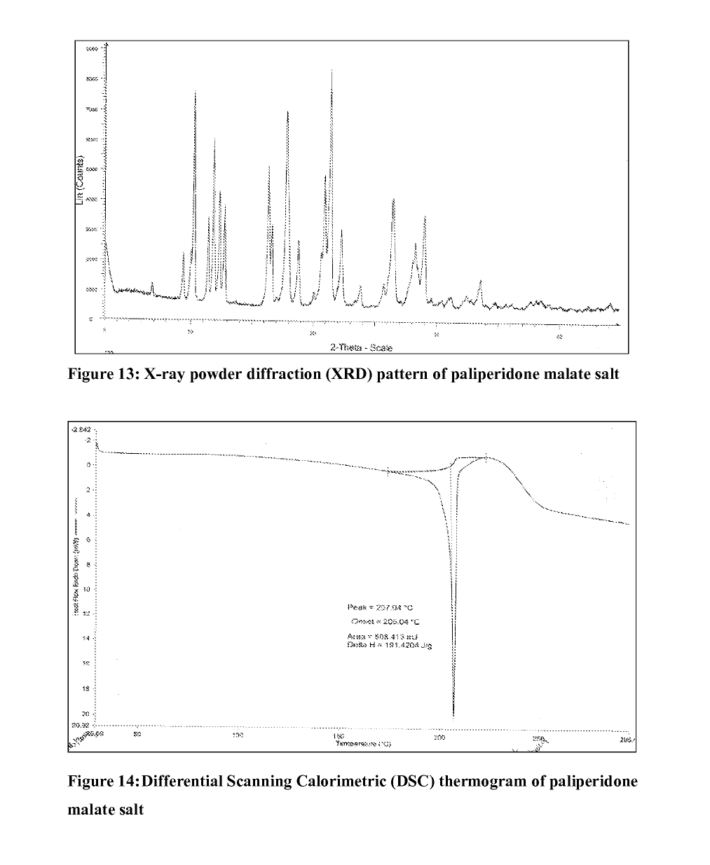

In one embodiment, the solid state forms of paliperidone salts have the following characteristics, wherein:
a) the solid state form of paliperidone L-(+)-tartrate salt is characterized by one or more of the following properties:
   i) a powder X-ray diffraction pattern substantially in accordance with FIG. 1;
   ii) a powder X-ray diffraction pattern having peaks at about 10.32, 11.88, 16.35, 17.93, 21.10 and 21.48±0.2 degrees 2-theta;
   iii) a powder X-ray diffraction pattern having additional peaks at about 11.43, 12.52, 12.88, 16.59, 18.15, 18.83, 20.61, 22.41, 23.93, 25.95, 26.45, 26.73, 28.25, 28.89, 29.13 and 33.56±0.2 degrees 2-theta; and
   iv) a differential scanning calorimetric (DSC) thermogram substantially in accordance with FIG. 2;
b) the solid state form of paliperidone tosylate salt is characterized by one or more of the following properties:
   i) a powder X-ray diffraction pattern substantially in accordance with FIG. 3;
   ii) a powder X-ray diffraction pattern having peaks at about 7.08, 8.83, 14.28, 15.26, 17.09, 18.69 and 23.49±0.2 degrees 2-theta;
   iii) a powder X-ray diffraction pattern having additional peaks at about 7.75, 11.24, 12.23, 13.59, 16.58, 17.75, 20.04, 21.24, 22.55, 24.29, 25.10 and 28.49±0.2 degrees 2-theta; and
   iv) a differential scanning calorimetric (DSC) thermogram substantially in accordance with FIG. 4;
c) the solid state form of paliperidone maleate salt is characterized by one or more of the following properties:
   i) a powder X-ray diffraction pattern substantially in accordance with FIG. 5;
   ii) a powder X-ray diffraction pattern having peaks at about 9.25, 11.02, 16.21, 16.47, 18.50 and 22.38±0.2 degrees 2-theta;
   iii) a powder X-ray diffraction pattern having additional peaks at about 6.82, 8.78, 11.67, 12.01, 14.98, 16.80, 19.38, 20.49, 20.85, 23.45, 24.92, 26.10, 27.07, 27.63, 28.09, 29.11 and 30.56±0.2 degrees 2-theta; and
   iv) a differential scanning calorimetric (DSC) thermogram substantially in accordance with FIG. 6;
d) the solid state form of paliperidone oxalate salt is characterized by one or more of the following properties:
   i) a powder X-ray diffraction pattern substantially in accordance with FIG. 7;
   ii) a powder X-ray diffraction pattern having peaks at about 13.32, 22.53, 23.78 and 27.73±0.2 degrees 2-theta;
   iii) a powder X-ray diffraction pattern having additional peaks at about 6.92, 11.60, 12.92, 15.32, 15.93, 16.60, 17.47, 19.22, 20.11, 24.74 and 27.18±0.2 degrees 2-theta; and
   iv) a differential scanning calorimetric (DSC) thermogram substantially in accordance with FIG. 8;
e) the solid state form of paliperidone acetate salt is characterized by one or more of the following properties:
   i) a powder X-ray diffraction pattern substantially in accordance with FIG. 11;
   ii) a powder X-ray diffraction pattern having peaks at about 8.16, 10.28, 13.78, 20.68, 24.66 and 25.06±0.2 degrees 2-theta;
   iii) a powder X-ray diffraction pattern having additional peaks at about 7.43, 13.12, 14.54, 14.94, 17.57, 18.63, 19.23, 20.04, 27.97, 30.95 and 31.22±0.2 degrees 2-theta; and
   iv) a differential scanning calorimetric (DSC) thermogram substantially in accordance with FIG. 12;
f) the solid state form of paliperidone malate salt is characterized by one or more of the following properties:
   i) a powder X-ray diffraction pattern substantially in accordance with FIG. 13;
   ii) a powder X-ray diffraction pattern having peaks at about 10.23, 11.86, 16.33, 17.82, 20.90, 21.40 and 26.46±0.2 degrees 2-theta;
   iii) a powder X-ray diffraction pattern having additional peaks at about 9.35, 9.97, 11.40, 12.32, 12.73, 16.58, 18.76, 2062, 22.25, 23.83, 25.71, 27.73, 28.03, 28.25, 28.97 and 33.58±0.2 degrees 2-theta; and
   iv) a differential scanning calorimetric (DSC) thermogram substantially in accordance with FIG. 14.

The solid state forms of paliperidone salts are stable, consistently reproducible, and are particularly suitable for bulk preparation and handling. Moreover, the solid state forms of paliperidone salts are useful intermediates in the preparation of paliperidone free base or a pharmaceutically acceptable salt in high purity.

According to another aspect, there is provided a process for the preparation of solid state form of a paliperidone salt, wherein the salt of paliperidone is an L-(+)-tartrate salt, a tosylate salt, a maleate salt, an oxalate salt, an acetate salt or a malate salt, comprising:
a) providing a first solution or a suspension of paliperidone free base in a first solvent;
b) combining the first solution or suspension with an acid to produce a second solution or suspension containing a paliperidone acid addition salt, wherein the acid is selected from the group consisting of L-(+)-tartaric acid, p-toluenesulfonic acid, maleic acid, oxalic acid, acetic acid and malic acid; and
c) optionally, substantially removing the solvent from the second solution or suspension to obtain a residue, followed by dissolving or suspending the residue in a second solvent to produce a third solution or suspension;
d) isolating and/or recovering the solid state form of paliperidone salt either from the second solution or suspension obtained in step-(b) or from the third solution or suspension obtained in step-(c).

The solid state form of paliperidone salt obtained by the process disclosed herein is further optionally converted into paliperidone free base or a pharmaceutically acceptable salt thereof by treating the solid state form of paliperidone salt with a base and/or an acid in a solvent.

The process can produce solid state forms of paliperidone salts in substantially pure form.

The term “substantially pure solid state form of paliperidone salt” refers to the solid state form of paliperidone salt having a purity of greater than about 98 wt %, specifically greater than about 99 wt %, more specifically greater than about 99.5 wt %, and still more specifically greater than about 99.9 wt %. The purity is preferably measured by High Performance Liquid Chromatography (HPLC). For example, the purity of solid state form of paliperidone salt obtained by the process disclosed herein can be about 98% to about 99.95%, or about 99% to about 99.99%, as measured by HPLC.

In one embodiment, the process disclosed herein provides stable solid state forms of paliperidone salts. The term “stable solid state form” refers to stability of the solid state form under the standard temperature and humidity conditions of testing of pharmaceutical products, wherein the stability is indicated by preservation of the original polymorphic form.

Exemplary first solvents used in step-(a) include, but are not limited to, water, an alcohol, a ketone, a chlorinated hydrocarbon, a hydrocarbon, an ester, a nitrile, an ether, a polar aprotic solvent, and mixtures thereof. The term solvent also includes mixtures of solvents.

In one embodiment, the first solvent is selected from the group consisting of water, methanol, ethanol, n-propanol, isopropyl alcohol, isobutanol, n-butanol, tert-butanol, amyl alcohol, isoamyl alcohol, hexanol, acetone, methyl ethyl ketone, methyl isobutyl ketone, methyl tert-butyl ketone, acetonitrile, ethyl acetate, methyl acetate, isopropyl acetate, tert-butyl methyl acetate, ethyl formate, methylene chloride, ethylene dichloride, chloroform, n-pentane, n-hexane, n-heptane, cyclohexane, toluene, xylene, tetrahydrofuran, dioxane, diethyl ether, diisopropyl ether, monoglyme, diglyme, N,N-dimethylformamide, N,N-dimethylacetamide, dimethylsulfoxide, and mixtures thereof.

Specifically, the first solvent is selected from the group consisting of water, methanol, ethanol, isopropyl alcohol, acetone, n-hexane, n-heptane, cyclohexane, and mixtures thereof; and more specifically water, ethanol, acetone, n-hexane, and mixtures thereof.

Step-(a) of providing a first solution of paliperidone free base includes dissolving paliperidone free base in the first solvent, or obtaining an existing solution from a previous processing step.

In one embodiment, the paliperidone is dissolved in the first solvent at a temperature of about 0° C. to the reflux temperature of the solvent used, specifically at about 25° C. to about 110° C., and more specifically at about 40° C. to about 80° C.

As used herein, “reflux temperature” means the temperature at which the solvent or solvent system refluxes or boils at atmospheric pressure.

In another embodiment, step-(a) of providing a suspension of paliperidone free base includes suspending paliperidone free base in the first solvent while stirring at a temperature of about 0° C. to the reflux temperature of the solvent used. In one embodiment, the suspension is stirred at a temperature of about 25° C. to about 110° C. for at least 30 minutes and more specifically at a temperature of about 40° C. to about 80° C. for about 1 hour to about 10 hours.

In another embodiment, the solution or suspension in step-(a) is prepared by reacting 3-(2-chloroethyl)-6,7,8,9-tetrahydro-9-hydroxy-2-methyl-4H-pyrido[1,2-a]-pyrimidin-4-one with 6-fluoro-3-(4-piperidinyl)-1,2-benzisoxazole in the presence of a base, optionally in the presence of a phase transfer catalyst, in a reaction inert solvent under suitable conditions to produce a reaction mass containing paliperidone free base, followed by usual work up such as washings, extractions, evaporations, filtrations, pH adjustments, or a combination thereof. In one embodiment, the work-up includes dissolving, suspending or extracting the resulting paliperidone in the first solvent at a temperature of about 0° C. to the reflux temperature of the solvent used, specifically at about 25° C. to about 110° C., and more specifically at about 40° C. to about 80° C.

Exemplary phase transfer catalysts suitable for facilitating the reaction between 3-(2-chloroethyl)-6,7,8,9-tetrahydro-9-hydroxy-2-methyl-4H-pyrido[1,2-a]-pyrimidin-4-one and 6-fluoro-3-(4-piperidinyl)-1,2-benzisoxazole include, but are not limited to, quaternary ammonium salts substituted with a group such as a straight or branched alkyl group having 1 to about 18 carbon atoms, a phenyl lower alkyl group including a straight or branched alkyl group having 1 to 6 carbon atoms which is substituted by an aryl group and phenyl group, e.g., tetrabutylammonium chloride, tetrabutylammonium bromide, tetrabutylammonium fluoride, tetrabutylammonium iodide, tetrabutylammonium hydroxide, tetrabutylammonium hydrogen sulfate, tributylmethylammonium chloride, tributylbenzylammonium chloride, tetraethylammonium chloride, tetramethylammonium chloride, tetrapentylammonium chloride, tetrapentylammonium bromide, tetrahexyl ammonium chloride, benzyldimethyloctylammonium chloride, methyltrihexylammonium chloride, benzylmethyloctadecanylammonium chloride, methyltridecanylammonium chloride, benzyltripropylammonium chloride, benzyltriethyl ammonium chloride, phenyltriethylammonium chloride and the like; phosphonium salts substituted with a residue such as a straight or branched alkyl group having 1 to about 18 carbon atoms, e.g., tetrabutylphosphonium chloride and the like; and pyridinium salts substituted with a straight or branched alkyl group having 1 to about 18 carbon atoms, e.g., 1-dodecanylpyridinium chloride and the like.

Specific phase transfer catalysts are tetrabutylammonium bromide, tetrabutylphosphonium bromide, tetrabutylammonium chloride, tetrabutylphosphonium chloride, benzyltriethylammonium chloride, tetrabutylammonium hydrogen sulfate, and more specifically tetrabutylammonium bromide.

Exemplary reaction inert solvents suitable for facilitating the reaction between 3-(2-chloroethyl)-6,7,8,9-tetrahydro-9-hydroxy-2-methyl-4H-pyrido[1,2-a]-pyrimidin-4-one and 6-fluoro-3-(4-piperidinyl)-1,2-benzisoxazole include, but are not limited to, water, an alcohol, a ketone, a cyclic ether, an aliphatic ether, a hydrocarbon, a chlorinated hydrocarbon, a nitrile, an ester, a polar aprotic solvent, and the like, and mixtures thereof. In one embodiment, the solvent is selected from the group consisting of water, methanol, ethanol, n-propanol, isopropanol, n-butanol, isobutanol, tert-butanol, amyl alcohol, hexanol, acetone, methyl ethyl ketone, methyl isobutyl ketone, methyl tert-butyl ketone, acetonitrile, ethyl acetate, methyl acetate, isopropyl acetate, tert-butyl methyl acetate, ethyl formate, dichloromethane, dichloroethane, chloroform, carbon tetrachloride, tetrahydrofuran, dioxane, diethyl ether, diisopropyl ether, monoglyme, diglyme, n-pentane, n-hexane, n-heptane, cyclohexane, toluene, xylene, N,N-dimethylformamide, N,N-dimethylacetamide, dimethylsulfoxide, and mixtures thereof.

In one embodiment, the base suitable for facilitating the reaction between 3-(2-chloroethyl)-6,7,8,9-tetrahydro-9-hydroxy-2-methyl-4H-pyrido[1,2-a]-pyrimidin-4-one and 6-fluoro-3-(4-piperidinyl)-1,2-benzisoxazole is an organic or inorganic base. Specific organic bases are triethyl amine, trimethylamine and N,N-diisopropylethylamine.

In another embodiment, the base is an inorganic base. Exemplary inorganic bases include, but are not limited to, hydroxides, alkoxides, carbonates and bicarbonates of alkali or alkaline earth metals, and ammonia. Specific inorganic bases are aqueous ammonia, sodium hydroxide, calcium hydroxide, magnesium hydroxide, potassium hydroxide, lithium hydroxide, sodium carbonate, potassium carbonate, lithium carbonate, sodium tert-butoxide, sodium isopropoxide and potassium tert-butoxide, and more specifically sodium hydroxide, potassium hydroxide, sodium carbonate and potassium carbonate.

Alternatively, the solution or suspension in step-(a) is prepared by treating an acid addition salt of paliperidone with a base to liberate paliperidone free base, followed by extracting, dissolving or suspending the paliperidone in the first solvent at a temperature of about 0° C. to the reflux temperature of the solvent used, specifically at about 25° C. to about 110° C., and more specifically at about 40° C. to about 80° C.

In another embodiment, the acid addition salt of paliperidone is derived from a therapeutically acceptable acid such as hydrochloric acid, acetic acid, propionic acid, sulfuric acid, nitric acid, succinic acid, maleic acid, fumaric acid, citric acid, glutaric acid, citraconic acid, glutaconic acid, tartaric acid, malic acid, and ascorbic acid. A specific salt is paliperidone hydrochloride.

The treatment of an acid addition salt with a base is carried out in a solvent and the selection of solvent is not critical. A wide variety of solvents such as chlorinated solvents, alcohols, ketones, hydrocarbon solvents, esters, ether solvents etc., can be used.

In one embodiment, the base used herein is an inorganic or an organic base selected from the group as described above.

The first solution or suspension obtained in step-(a) is optionally stirred at a temperature of about 25° C. to the reflux temperature of the solvent used for at least 15 minutes, and specifically at a temperature of about 40° C. to the reflux temperature of the solvent used for about 20 minutes to about 8 hours.

The acid in step-(b) may be used directly or in the form of a solution containing the acid and a suitable solvent. The solvent used for diluting the acid is selected from the group consisting of water, methanol, ethanol, n-propanol, isopropyl alcohol, isobutanol, n-butanol, tert-butanol, amyl alcohol, isoamyl alcohol, hexanol, acetone, methyl ethyl ketone, methyl isobutyl ketone, methyl tert-butyl ketone, acetonitrile, ethyl acetate, methyl acetate, isopropyl acetate, tert-butyl methyl acetate, ethyl formate, methylene chloride, ethylene dichloride, chloroform, n-pentane, n-hexane, n-heptane, cyclohexane, toluene, xylene, tetrahydrofuran, dioxane, diethyl ether, diisopropyl ether, monoglyme, diglyme, N,N-dimethylformamide, N,N-dimethylacetamide, dimethylsulfoxide, and mixtures thereof.

Combining of the first solution or suspension with acid in step-(b) is done in a suitable order, for example, the first solution or suspension is added to the acid, or alternatively, the acid is added to the first solution or suspension. The addition is, for example, carried out drop wise or in one portion or in more than one portion. The addition is specifically carried out at a temperature of about 0° C. to the reflux temperature of the solvent used, more specifically at about 25° C. to about 110° C., and most specifically at about 40° C. to about 80° C. under stirring. After completion of addition process, the resulting mass is stirred at a temperature of about 0° C. to the reflux temperature of the solvent used for at least 10 minutes, specifically at about 25° C. to about 110° C. for about 20 minutes to about 25 hours, and more specifically at a temperature of about 40° C. to about 80° C. for about 30 minutes to about 8 hours to produce a second solution or suspension.

The second solution obtained in step-(b) is optionally subjected to carbon treatment or silica gel treatment. The carbon treatment or silica gel treatment is carried out by methods known in the art, for example, by stirring the solution with finely powdered carbon or silica gel at a temperature of below about 80° C. for at least 15 minutes, specifically at a temperature of about 40° C. to about 70° C. for at least 30 minutes; and filtering the resulting mixture through hyflo to obtain a filtrate containing paliperidone acid addition salt by removing charcoal or silica gel. Specifically, the finely powdered carbon is an active carbon. A specific mesh size of silica gel is 40-500 mesh, and more specifically 60-120 mesh.

The term “substantially removing” the solvent refers to at least 30%, specifically greater than about 50%, more specifically greater than about 90%, still more specifically greater than about 99%, and most specifically essentially complete (100%), removal of the solvent from the solvent solution.

Removal of solvent in step-(c) is accomplished, for example, by substantially complete evaporation of the solvent, concentrating the solution or distillation of solvent under inert atmosphere, or a combination thereof, to substantial elimination of total solvent present in the reaction mass.

The distillation process can be performed at atmospheric pressure or reduced pressure. Specifically, the distillation is carried out at a temperature of about 30° C. to about 110° C., more specifically at about 40° C. to about 90° C., and most specifically at about 45° C. to about 80° C.

Specifically, the solvent is removed at a pressure of about 760 mm Hg or less, more specifically at about 400 mm Hg or less, still more specifically at about 80 mm Hg or less, and most specifically from about 30 to about 80 mm Hg.

The residue containing paliperidone acid addition salt obtained in step-(c) is dissolved or suspended in the second solvent a temperature of about 0° C. to the reflux temperature of the solvent used, specifically at about 20° C. to about 110° C., and more specifically at about 25° C. to about 80° C. In one embodiment, the solution or suspension is stirred at a temperature of about 20° C. to about 110° C. for at least 10 minutes and more specifically at a temperature of about 25° C. to about 80° C. for about 20 minutes to about 10 hours.

Exemplary second solvents used in step-(c) include, but are not limited to, water, an alcohol, a ketone, a chlorinated hydrocarbon, a hydrocarbon, an ester, a nitrile, an ether, a polar aprotic solvent, and mixtures thereof. The term solvent also includes mixtures of solvents.

In one embodiment, the second solvent is selected from the group consisting of water, methanol, ethanol, n-propanol, isopropyl alcohol, isobutanol, n-butanol, tert-butanol, amyl alcohol, isoamyl alcohol, hexanol, acetone, methyl ethyl ketone, methyl isobutyl ketone, methyl tert-butyl ketone, acetonitrile, ethyl acetate, methyl acetate, isopropyl acetate, tert-butyl methyl acetate, ethyl formate, methylene chloride, ethylene dichloride, chloroform, n-pentane, n-hexane, n-heptane, cyclohexane, toluene, xylene, tetrahydrofuran, dioxane, diethyl ether, diisopropyl ether, monoglyme, diglyme, N,N-dimethylformamide, N,N-dimethylacetamide, dimethylsulfoxide, and mixtures thereof.

Specifically, the second solvent is selected from the group consisting of tetrahydrofuran, dioxane, diethyl ether, diisopropyl ether, monoglyme, diglyme, and mixtures thereof; and more specifically diethyl ether and diisopropyl ether.

The isolation of pure solid state form of paliperidone salt in step-(d) is carried out by forcible crystallization, spontaneous crystallization, substantial removal of the solvent from the solution or suspension, or a combination thereof.

Spontaneous crystallization refers to crystallization without the help of an external aid such as seeding, cooling etc., and forcible crystallization refers to crystallization with the help of an external aid.

Forcible crystallization may be initiated by a method usually known in the art such as cooling, seeding, partial removal of the solvent from the solution, by adding an anti-solvent to the solution, or a combination thereof.

The term “anti-solvent” refers to a solvent which when added to an existing solution of a substance reduces the solubility of the substance.

Exemplary anti-solvents include, but are not limited to, water, an alcohol, a ketone, a chlorinated hydrocarbon, a hydrocarbon, an ester, a nitrile, an ether, a polar aprotic solvent, and mixtures thereof. Specifically, the anti-solvent is selected from the group consisting of an alcohol, a hydrocarbon, an ether, and mixtures thereof and more specifically, the anti-solvent is selected from the group consisting of methanol, ethanol, n-propanol, isopropyl alcohol, isobutanol, n-butanol, tert-butanol, amyl alcohol, isoamyl alcohol, hexanol, n-pentane, n-hexane, n-heptane, cyclohexane, toluene, xylene, tetrahydrofuran, dioxane, diethyl ether, diisopropyl ether, monoglyme, diglyme, and mixtures thereof.

In one embodiment, the crystallization is carried out by cooling the solution under stirring at a temperature of below 30° C. for at least 10 minutes, specifically at about 0° C. to about 30° C. for about 30 minutes to about 20 hours.

Removal of solvent is accomplished, for example, by substantially complete evaporation of the solvent, concentrating the solution or distillation of solvent, under inert atmosphere to obtain solid state form of paliperidone salt.

In one embodiment, the solvent is removed by evaporation. Evaporation can be achieved at sub-zero temperatures by lyophilisation or freeze-drying techniques. The solution may also be completely evaporated in, for example, a pilot plant Rota vapor, a Vacuum Paddle Dryer or in a conventional reactor under vacuum above about 720 mm Hg by flash evaporation techniques by using an agitated thin film dryer (“ATFD”), or evaporated by spray drying to obtain a dry amorphous powder.

The distillation process can be performed at atmospheric pressure or reduced pressure. Specifically, the solvent is removed at a pressure of about 760 mm Hg or less, more specifically at about 400 mm Hg or less, still more specifically at about 80 mm Hg or less, and most specifically from about 30 to about 80 mm Hg.

Solvents can also be removed by spray-drying, in which a solution of paliperidone salt is sprayed into the spray drier at the flow rate ranging from 10 to 300 ml/hr, specifically 40 to 200 ml/hr. The air inlet temperature to the spray drier used may range from about 30° C. to about 150° C., specifically from about 65° C. to about 110° C. and the outlet air temperature used may range from about 30° C. to about 90° C.

Another suitable method is vertical agitated thin-film drying (or evaporation). Agitated thin film evaporation technology involves separating the volatile component using indirect heat transfer coupled with mechanical agitation of the flowing film under controlled conditions. In vertical agitated thin-film drying (or evaporation) (ATFD-V), the starting solution is fed from the top into a cylindrical space between a centered rotary agitator and an outside heating jacket. The rotor rotation agitates the downside-flowing solution while the heating jacket heats it.

The recovering in step-(d) is carried out by methods such as filtration, filtration under vacuum, decantation, centrifugation, or a combination thereof. In one embodiment, solid state form of paliperidone salt is recovered by filtration employing a filtration media of, for example, a silica gel or celite.

The substantially pure solid state form of paliperidone salt obtained by above process may be further dried in, for example, a Vacuum Tray Dryer, a Rotocon Vacuum Dryer, a Vacuum Paddle Dryer or a pilot plant Rota vapor, to further lower residual solvents. Drying can be carried out under reduced pressure until the residual solvent content reduces to the desired amount such as an amount that is within the limits given by the International Conference on Harmonization of Technical Requirements for Registration of Pharmaceuticals for Human Use (“ICH”) guidelines.

In one embodiment, the drying is carried out at atmospheric pressure or reduced pressures, such as below about 200 mm Hg, or below about 50 mm Hg, at temperatures such as about 35° C. to about 90° C. The drying can be carried out for any desired time period that achieves the desired result, such as about 1 to 20 hours. Drying may also be carried out for shorter or longer periods of time depending on the product specifications. Temperatures and pressures will be chosen based on the volatility of the solvent being used and the foregoing should be considered as only a general guidance. Drying can be suitably carried out in a tray dryer, vacuum oven, air oven, or using a fluidized bed drier, spin flash dryer, flash dryer and the like. Drying equipment selection is well within the ordinary skill in the art.

The purity of the solid state form of paliperidone salt obtained by the process disclosed herein is greater than about 98%, specifically greater than about 99%, more specifically greater than about 99.9%, and most specifically greater than about 99.95% as measured by HPLC. For example, the purity of the solid state form of paliperidone salt can be about 99% to about 99.95%, or about 99.5% to about 99.99%.

Paliperidone and pharmaceutically acceptable salts of paliperidone can be prepared in high purity by using the substantially pure solid state forms of paliperidone salts obtained according to the process disclosed herein.

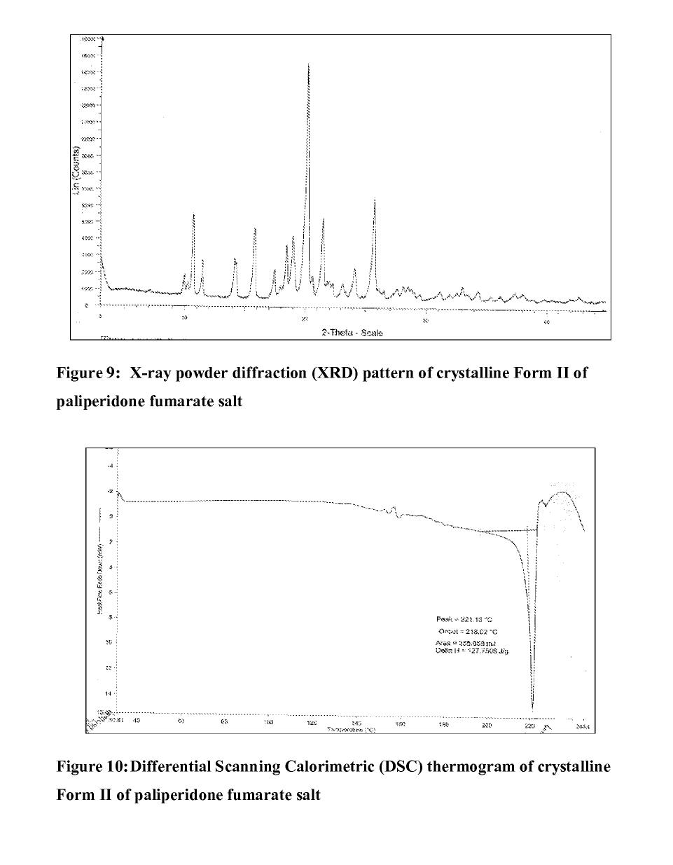

According to another aspect, there is provided a novel crystalline form of paliperidone fumarate, designated as paliperidone fumarate crystalline Form II, characterized by one or more of the following properties:
i) a powder X-ray diffraction pattern substantially in accordance with FIG. 9;
ii) a powder X-ray diffraction pattern having peaks at about 10.65, 14.16, 15.79, 19.87, 20.17, 21.45 and 25.70±0.2 degrees 2-theta;
iii) a powder X-ray diffraction pattern having additional peaks at about 9.93, 11.43, 17.43, 18.99, 20.58 and 24.09±0.2 degrees 2-theta;
iv) a powder X-ray diffraction pattern having no peaks at about 7.60, 8.21, 13.81, 15.23, 26.98 and 29.11±0.2 degrees 2-theta; and
v) a differential scanning calorimetric (DSC) thermogram substantially in accordance with FIG. 10.

The paliperidone fumarate crystalline Form II is stable, consistently reproducible, and is particularly suitable for bulk preparation and handling. Moreover, the crystalline Form II of paliperidone fumarate is useful intermediate in the preparation of paliperidone in high purity.

The crystalline Form II of paliperidone fumarate has good flow properties and stable at room temperature, enhanced temperature, at relative high humidities, and in aqueous media.

According to another aspect, there is provided a process for the preparation of paliperidone fumarate crystalline Form II, comprising:
a) providing a suspension of paliperidone free base in ethanol;
b) combining the suspension with fumaric acid to produce a reaction mass containing paliperidone fumarate; and
c) isolating and/or recovering the crystalline Form II of paliperidone fumarate from the reaction mass obtained in step-(b).

In one embodiment, the process steps-(a), (b) and (c) are, each independently, carried out by the methods as described hereinabove.

According to another aspect, there is provided a process for preparing highly pure paliperidone free base, comprising:
a) contacting solid state form of a paliperidone salt with a base in a first solvent to provide a reaction mass containing paliperidone free base, wherein the salt of paliperidone is an L-(+)-tartrate salt, a tosylate salt, a maleate salt, an oxalate salt, an acetate salt or a malate salt; and
b) optionally, recovering the paliperidone free base from the reaction mass obtained in step-(a) and followed by extracting, suspending or dissolving the paliperidone free base in a second solvent;
c) isolating and/or recovering the pure paliperidone free base either from the reaction mass obtained in step-(a) or from the solution or suspension obtained in step-(b).

In one embodiment, the process disclosed herein or any one of the process steps can be repeated any number of times to provide paliperidone free base with the desired purity.

Exemplary first solvents used in step-(a) include, but are not limited to, water, an alcohol, a ketone, a chlorinated hydrocarbon, a hydrocarbon, an ester, a nitrile, an ether, a polar aprotic solvent, an organosulfur solvent, and mixtures thereof. The term solvent also includes mixtures of solvents.

In one embodiment, the first solvent is selected from the group consisting of water, methanol, ethanol, n-propanol, isopropyl alcohol, isobutanol, n-butanol, tert-butanol, amyl alcohol, isoamyl alcohol, hexanol, acetone, methyl ethyl ketone, methyl isobutyl ketone, methyl tert-butyl ketone, acetonitrile, ethyl acetate, methyl acetate, isopropyl acetate, tert-butyl methyl acetate, ethyl formate, methylene chloride, ethylene dichloride, chloroform, n-pentane, n-hexane, n-heptane, cyclohexane, toluene, xylene, tetrahydrofuran, dioxane, diethyl ether, diisopropyl ether, monoglyme, diglyme, N,N-dimethylformamide, N,N-dimethylacetamide, dimethylsulfoxide, sulfolane, and mixtures thereof.

Specifically, the first solvent is selected from the group consisting of water, methanol, ethanol, isopropyl alcohol, acetone, sulfolane, and mixtures thereof; and more specifically water, methanol, sulfolane, and mixtures thereof.

In one embodiment, the base used in step-(a) is an organic or inorganic base selected from the group as described above.

In one embodiment, the contacting in step-(a) is carried out under stirring at a temperature of below about reflux temperature of the solvent used for at least 10 minutes, specifically at a temperature of about 0° C. to about 80° C. for about 15 minutes to about 15 hours, and more specifically at about 20° C. to about 60° C. for about 20 minutes to about 5 hours. In another embodiment, the pH of the reaction mass is adjusted between 9 and 12 during the addition of base.

The recovering in step-(b) is carried out by the methods as described above.

Exemplary second solvents used in step-(b) include, but are not limited to, water, an alcohol, a ketone, a chlorinated hydrocarbon, a hydrocarbon, an ester, a nitrile, an ether, a polar aprotic solvent, an organosulfur solvent, and mixtures thereof. The term solvent also includes mixtures of solvents.

In one embodiment, the second solvent is selected from the group as described above. Specifically, the second solvent is selected from the group consisting of methanol, ethanol, n-propanol, isopropyl alcohol, acetone, sulfolane, and mixtures thereof and more specifically methanol, acetone, sulfolane, and mixtures thereof.

In one embodiment, the paliperidone free base in step-(b) is dissolved in the second solvent at a temperature of about 0° C. to the reflux temperature of the solvent used, specifically at about 25° C. to about 110° C., and more specifically at about 40° C. to about 80° C.

In another embodiment, the paliperidone free base is suspended in the second solvent while stirring at a temperature of about 0° C. to the reflux temperature of the solvent used. In one embodiment, the suspension is stirred at a temperature of about 0° C. to the reflux temperature of the solvent used for at least 30 minutes, and more specifically at a temperature of about 25° C. to about 110° C. for about 1 hour to about 10 hours.

The isolation of pure paliperidone free base in step-(c) is carried out by the methods as described above.

In one embodiment, the crystallization is carried out by cooling the solution under stirring at a temperature of below 30° C. for at least 10 minutes, specifically at about 0° C. to about 30° C. for about 30 minutes to about 20 hours.

The pure paliperidone free base obtained by above process is recovered and optionally further dried as described above.

The purity of the paliperidone free base obtained by the process disclosed herein is of greater than about 99%, specifically greater than about 99.5%, more specifically greater than about 99.9%, and most specifically greater than about 99.95% as measured by HPLC. For example, the purity of the paliperidone free base can be about 99% to about 99.95%, or about 99.5% to about 99.99%.

Further encompassed herein is the use of the solid state form of a paliperidone salt for the manufacture of a pharmaceutical composition together with a pharmaceutically acceptable carrier, wherein the salt of paliperidone is an L-(+)-tartrate salt, a tosylate salt, a maleate salt, an oxalate salt, an acetate salt or a malate salt.

A specific pharmaceutical composition of the solid state form of paliperidone salt is selected from a solid dosage form and an oral suspension.

In one embodiment, the solid state form of paliperidone salt has a D₉₀ particle size of less than or equal to about 500 microns, specifically about 1 micron to about 300 microns, and most specifically about 10 microns to about 150 microns, wherein the salt of paliperidone is an L-(+)-tartrate salt, a tosylate salt, a maleate salt, an oxalate salt, an acetate salt or a malate salt.

In another embodiment, the particle sizes of the solid state form of paliperidone salt are produced by a mechanical process of reducing the size of particles which includes any one or more of cutting, chipping, crushing, milling, grinding, micronizing, trituration or other particle size reduction methods known in the art, to bring the solid state form to the desired particle size range.

According to another aspect, there is provided pharmaceutical compositions comprising the solid state form of paliperidone salt and one or more pharmaceutically acceptable excipients, wherein the salt of paliperidone is an L-(+)-tartrate salt, a tosylate salt, a maleate salt, an oxalate salt, an acetate salt or a malate salt.

According to another aspect, there is provided pharmaceutical compositions comprising the solid state form of paliperidone salt prepared according to process disclosed herein and one or more pharmaceutically acceptable excipients, wherein the salt of paliperidone is an L-(+)-tartrate salt, a tosylate salt, a maleate salt, an oxalate salt, an acetate salt or a malate salt.

According to another aspect, there is provided a process for preparing a pharmaceutical formulation comprising combining the solid state form of paliperidone salt prepared according to processes disclosed herein, with one or more pharmaceutically acceptable excipients, wherein the salt of paliperidone is an L-(+)-tartrate salt, a tosylate salt, a maleate salt, an oxalate salt, an acetate salt or a malate salt.

According to another aspect, there is provided a method for treating a patient suffering from psychotic diseases; comprising administering a solid state form of paliperidone salt, or a pharmaceutical composition that comprises the solid state form of paliperidone salt along with pharmaceutically acceptable excipients, wherein the salt of paliperidone is an L-(+)-tartrate salt, a tosylate salt, a maleate salt, an oxalate salt, an acetate salt or a malate salt.

Yet in another embodiment, pharmaceutical compositions comprise at least a therapeutically effective amount of solid state form of a paliperidone salt, wherein the salt of paliperidone is an L-(+)-tartrate salt, a tosylate salt, a maleate salt, an oxalate salt, an acetate salt or a malate salt. Such pharmaceutical compositions may be administered to a mammalian patient in a dosage form, e.g., solid, liquid, powder, elixir, aerosol, syrups, injectable solution, etc. Dosage forms may be adapted for administration to the patient by oral, buccal, parenteral, ophthalmic, rectal and transdermal routes or any other acceptable route of administration. Oral dosage forms include, but are not limited to, tablets, pills, capsules, syrup, troches, sachets, suspensions, powders, lozenges, elixirs and the like. The solid state form of paliperidone salt may also be administered as suppositories, ophthalmic ointments and suspensions, and parenteral suspensions, which are administered by other routes, wherein the salt of paliperidone is an L-(+)-tartrate salt, a tosylate salt, a maleate salt, an oxalate salt, an acetate salt or a malate salt.

The pharmaceutical compositions further contain one or more pharmaceutically acceptable excipients. Suitable excipients and the amounts to use may be readily determined by the formulation scientist based upon experience and consideration of standard procedures and reference works in the field, e.g., the buffering agents, sweetening agents, binders, diluents, fillers, lubricants, wetting agents and disintegrants described herein.

In one embodiment, capsule dosage forms contain solid state form of paliperidone salt within a capsule which may be coated with gelatin. Tablets and powders may also be coated with an enteric coating. Suitable enteric coating agents include phthalic acid cellulose acetate, hydroxypropylmethyl cellulose phthalate, polyvinyl alcohol phthalate, carboxy methyl ethyl cellulose, a copolymer of styrene and maleic acid, a copolymer of methacrylic acid and methyl methacrylate, and like materials, and if desired, the coating agents may be employed with suitable plasticizers and/or extending agents. A coated capsule or tablet may have a coating on the surface thereof or may be a capsule or tablet comprising a powder or granules with an enteric-coating.

Tableting compositions may have few or many components depending upon the tableting method used, the release rate desired and other factors. For example, the compositions described herein may contain diluents such as cellulose-derived materials such as powdered cellulose, microcrystalline cellulose, microtine cellulose, methyl cellulose, ethyl cellulose, hydroxyethyl cellulose, hydroxypropyl cellulose, hydroxypropylmethyl cellulose, carboxymethyl cellulose salts and other substituted and unsubstituted celluloses; starch; pregelatinized starch; inorganic diluents such calcium carbonate and calcium diphosphate and other diluents known to one of ordinary skill in the art. Yet other suitable diluents include waxes, sugars (e.g. lactose) and sugar alcohols such as mannitol and sorbitol, acrylate polymers and copolymers, as well as pectin, dextrin and gelatin.

Other excipients include binders, such as acacia gum, pregelatinized starch, sodium alginate, glucose and other binders used in wet and dry granulation and direct compression tableting processes; disintegrants such as sodium starch glycolate, crospovidone, low-substituted hydroxypropyl cellulose and others; lubricants like magnesium and calcium stearate and sodium stearyl fumarate; flavorings; sweeteners; preservatives; pharmaceutically acceptable dyes and glidants such as silicon dioxide.

### Instrumental Details:

### High Pressure Liquid Chromatography:

The HPLC purity was measured by high performance liquid chromatography by using Waters, alliance 2695 HPLC system having dual wavelength and 2487 UV detector under the following conditions:

### Column: ACE-3-C18, 150*4.6 mm, Part Number-ACE-111-1546.

Column oven temperature: 25° C.

### Detection: UV at 237 nm and 210 nm

Flow rate: 10 mL/minute
Injection volume: 10 μL
Run time: 55 minutes
Diluents: Water:acetonitrile (50:50 v/v)
Sample concentration: Prepare a mixture of 2.0 mg/ml of sample in diluents.

### X-Ray Powder Diffraction (P-XRD):

The X-Ray powder diffraction was measured by an X-ray powder diffractometer equipped with a Cu-anode (λ=1.54 Angstrom), X-ray source was operated at 40 kV, 40 mA and a Ni filter was used to strip K-beta radiation. Two-theta calibration was performed using an NIST SRM 1976, Corundum standard. The sample was analyzed using the following instrument parameters: measuring range=3-45° 2-theta; step width=0.01579°; and measuring time per step=0.11 second.

### Differential Scanning Calorimetry (DSC):

Differential Scanning calorimetry (DSC) measurements were performed with a Differential Scanning calorimeter (DSC Q 1000 V23.5 Build 72, Universal V4.3A TA Instruments) at a scan rate of 5° C. per minute.

The following examples are given for the purpose of illustrating the present disclosure and should not be considered as limitation on the scope or spirit of the disclosure.

### EXAMPLES

### Example 1

### Preparation of Paliperidone Tartrate

Paliperidone (2.5 g) was suspended in ethanol (50 ml) at 25-30° C., followed by heating at reflux temperature. This process was followed by the slow addition of a solution of L-tartaric acid (1.76 g) in ethanol (25 ml) at reflux temperature to form a clear solution. The solution was maintained at reflux for a further 15 minutes whereby precipitation started. The resulting suspension was cooled to 25-30° C., and then stirred for overnight at this temperature. The precipitated product was collected by filtration, washed with ethanol (5 ml), and then dried to give 2.89 g of paliperidone tartrate as off-white powder.

### Example 2

### Preparation of Paliperidone Tosylate

Paliperidone (2.5 g) was suspended in ethanol (50 ml) at 25-30° C. and heated at reflux temperature. This process was followed by the slow addition of a solution of p-toluenesulfonic acid (2.47 g) in ethanol (12 ml) at reflux temperature. The resulting clear solution was maintained at reflux for a further 15 minutes. The resulting mass was cooled to 25-30° C. and the suspension was stirred for 22 hours at this temperature. The solvent was evaporated and the residue was diluted with diethyl ether (50 ml) and then stirred for 1 hour at 25-30° C. The precipitated product was collected by filtration, washed with diethyl ether (10 ml), and then dried to give 3.0 g of paliperidone tosylate as off-white powder.

### Example 3

### Preparation of Paliperidone Maleate

Paliperidone (2.5 g) was suspended in ethanol (45 ml) at 25-30° C. and heated at 63-65° C. A solution of maleic acid (0.681 g) in ethanol (5 ml) was slowly added to the suspension at 63-65° C. for 10 minutes. The clear solution was maintained at 63-65° C. for further 15 minutes whereby precipitation started. The resulting suspension was cooled to 25-30° C. followed by stirring the suspension for 15 hours at 25-30° C. The precipitated product was collected by filtration, washed with ethanol (5 ml) and then dried to give 2.5 g of the paliperidone maleate as off-white powder.

### Example 4

### Preparation of Paliperidone Oxalate

Paliperidone (2.5 g) was suspended in ethanol (38 ml) at 25-30° C. and the suspension was heated at 70° C. A solution of oxalic acid (0.739 g) in ethanol (25 ml) was added to the suspension at 70° C. and the resulting clear solution was maintained at 70° C. for a further 15 minutes whereby precipitation started. The resulting suspension was cooled to 25-30° C. and then stirred for 6 hours at 25-30° C. The precipitated product was collected by filtration, washed with ethanol (5 ml) and then dried to give 2.87 g of paliperidone oxalate as off-white powder.

### Example 5

### Preparation of Paliperidone Fumarate Crystalline Form II

Paliperidone (2.5 g) was suspended in ethanol (38 ml) at 25-30° C. and the suspension heated at 70° C. A solution of fumaric acid (0.681 g) in ethanol (25 ml) was added to the suspension at 70° C. and the resulting clear solution was maintained at 70° C. for further 15 minutes whereby precipitation started. The resulting suspension was cooled to 25-30° C. and stirred the suspension for 6 hours at 25-30° C. The precipitated product was collected by filtration, washed with ethanol (5 ml) and then dried to give 2.41 g of paliperidone fumarate as off-white powder.

### Example 6

### Preparation of Paliperidone Acetate

Paliperidone (2.5 g) was added to a mixture of n-hexane (7.5 ml), acetone (0.25 ml), water (0.1 ml) and acetic acid (0.35 g) at 25-30° C. The resulting mixture was stirred for 30 minutes at 25-30° C. The upper layer was decanted from the mass and ethanol (30 ml) was added to the lower layer followed by stirring for 2 hours at 25-30° C. whereby precipitation started. The precipitated product was collected by filtration, washed with ethanol (5 ml) and dried to give 2.1 g of paliperidone acetate as off-white powder.

### Example 7

### Preparation of Paliperidone Malate

Paliperidone (2.5 g) was suspended in ethanol (75 ml) at 25-30° C., followed by heating the suspension at reflux temperature. Malic acid (0.786 g) was added to the suspension and the resulting clear solution was maintained at reflux temperature for further 15 minutes where by precipitation started. The resulting suspension was cooled to 25-30° C. and stirred the suspension for 6 hours at this temperature. The precipitated product was collected by filtration, washed with ethanol (5 ml) and dried to give 2.70 g of paliperidone malate as off-white powder.

### Example 8

### Preparation of Pure Paliperidone Free Base

Paliperidone oxalate (1.0 g) was added to water (5 ml) at 25-30° C., followed by pH adjustment to 10 to 11 using dilute aqueous ammonia solution at 25-30° C. The resulting mass was stirred for 1 hour 30 minutes. The solid product was collected by filtration, washed with water (2×5 ml) and acetone (2×5 ml). The resulting wet cake was suction dried and then suspended in acetone (10 ml) followed by reflux of slurry for 30 minutes. The resulting slurry was cooled, filtered and the wet cake was washed with acetone (5 ml). The wet material was dried in the oven at 45-50° C. under reduced pressure to yield 0.7 g of pure paliperidone.

### Example 9

### Solubility Data of Paliperidone and its Salts

The solubility data of paliperidone and solid state forms of paliperidone salts is furnished in the below table. The 100 mg of sample was used for solubility test.

| | | Solubility | |
|---|---|---|---|
| Sr. No. | Sample Name | Water | Methanol |
| 1. | Paliperidone | Insoluble | Insoluble |
| 2. | Paliperidone tartrate | Insoluble | Insoluble |
| 3. | Paliperidone tosylate | 100 mg/10 ml | 100 mg/1 ml |
| | | (sonication) | |
| 4. | Paliperidone maleate | 100 mg/1 ml | 100 mg/10 ml |
| 5. | Paliperidone oxalate | 100 mg/5 ml | 100 mg/15 ml |
| 6. | Paliperidone fumarate | 100 mg/25 ml | Insoluble |
| | crystalline Form II | (sonication) | |
| 7. | Paliperidone acetate | Insoluble | Insoluble |
| 8. | Paliperidone malate | 100 mg/15 ml | Insoluble |

Unless otherwise indicated, the following definitions are set forth to illustrate and define the meaning and scope of the various terms used to describe the invention herein.

The term “solid state form of paliperidone salts disclosed herein” includes crystalline forms, amorphous forms, hydrated, and solvated forms of paliperidone salts.

The term “crystalline form” refers to a crystal modification that can be characterized by analytical methods such as X-ray powder diffraction, IR-spectroscopy, differential scanning calorimetry (DSC) or by its melting point.

The term “pharmaceutically acceptable” means that which is useful in preparing a pharmaceutical composition that is generally non-toxic and is not biologically undesirable, and includes that which is acceptable for veterinary use and/or human pharmaceutical use.

The term “pharmaceutical composition” is intended to encompass a drug product including the active ingredient(s), pharmaceutically acceptable excipients that make up the carrier, as well as any product which results, directly or indirectly, from combination, complexation or aggregation of any two or more of the ingredients. Accordingly, the pharmaceutical compositions encompass any composition made by admixing the active ingredient, active ingredient dispersion or composite, additional active ingredient(s), and pharmaceutically acceptable excipients.

The term “therapeutically effective amount” as used herein means the amount of a compound that, when administered to a mammal for treating a state, disorder or condition, is sufficient to effect such treatment. The “therapeutically effective amount” will vary depending on the compound, the disease and its severity and the age, weight, physical condition and responsiveness of the mammal to be treated.

The term “delivering” as used herein means providing a therapeutically effective amount of an active ingredient to a particular location within a host causing a therapeutically effective blood concentration of the active ingredient at the particular location. This can be accomplished, e.g., by topical, local or by systemic administration of the active ingredient to the host.

The term “buffering agent” as used herein is intended to mean a compound used to resist a change in pH upon dilution or addition of acid of alkali. Such compounds include, by way of example and without limitation, potassium metaphosphate, potassium phosphate, monobasic sodium acetate and sodium citrate anhydrous and dehydrate and other such materials known to those of ordinary skill in the art.

The term “sweetening agent” as used herein is intended to mean a compound used to impart sweetness to a formulation. Such compounds include, by way of example and without limitation, aspartame, dextrose, glycerin, mannitol, saccharin sodium, sorbitol, sucrose, fructose and other such materials known to those of ordinary skill in the art.

The term “binders” as used herein is intended to mean substances used to cause adhesion of powder particles in granulations. Such compounds include, by way of example and without limitation, acacia, alginic acid, tragacanth, carboxymethylcellulose sodium, polyvinylpyrrolidone, compressible sugar (e.g., NuTab), ethylcellulose, gelatin, liquid glucose, methylcellulose, pregelatinized starch, starch, polyethylene glycol, guar gum, polysaccharide, bentonites, sugars, invert sugars, poloxamers (PLURONIC™ F68, PLURONIC™ F127), collagen, albumin, celluloses in non-aqueous solvents, polypropylene glycol, polyoxyethylene-polypropylene copolymer, polyethylene ester, polyethylene sorbitan ester, polyethylene oxide, microcrystalline cellulose, combinations thereof and other material known to those of ordinary skill in the art.

The term “diluent” or “filler” as used herein is intended to mean inert substances used as fillers to create the desired bulk, flow properties, and compression characteristics in the preparation of solid dosage formulations. Such compounds include, by way of example and without limitation, dibasic calcium phosphate, kaolin, sucrose, mannitol, microcrystalline cellulose, powdered cellulose, precipitated calcium carbonate, sorbitol, starch, combinations thereof and other such materials known to those of ordinary skill in the art.

The term “glidant” as used herein is intended to mean agents used in solid dosage formulations to improve flow-properties during tablet compression and to produce an anti-caking effect. Such compounds include, by way of example and without limitation, colloidal silica, calcium silicate, magnesium silicate, silicon hydrogel, cornstarch, talc, combinations thereof and other such materials known to those of ordinary skill in the art.

The term “lubricant” as used herein is intended to mean substances used in solid dosage formulations to reduce friction during compression of the solid dosage. Such compounds include, by way of example and without limitation, calcium stearate, magnesium stearate, mineral oil, stearic acid, zinc stearate, combinations thereof and other such materials known to those of ordinary skill in the art.

The term “disintegrant” as used herein is intended to mean a compound used in solid dosage formulations to promote the disruption of the solid mass into smaller particles which are more readily dispersed or dissolved. Exemplary disintegrants include, by way of example and without limitation, starches such as corn starch, potato starch, pregelatinized, sweeteners, clays, such as bentonite, microcrystalline cellulose (e.g., Avicel™), carsium (e.g., Amberlite™), alginates, sodium starch glycolate, gums such as agar, guar, locust bean, karaya, pectin, tragacanth, combinations thereof and other such materials known to those of ordinary skill in the art.

The term “wetting agent” as used herein is intended to mean a compound used to aid in attaining intimate contact between solid particles and liquids. Exemplary wetting agents include, by way of example and without limitation, gelatin, casein, lecithin (phosphatides), gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters, (e.g., TWEEN™s), polyethylene glycols, polyoxyethylene stearates colloidal silicon dioxide, phosphates, sodium dodecylsulfate, carboxymethylcellulose calcium, carboxymethylcellulose sodium, methylcellulose, hydroxyethylcellulose, hydroxylpropylcellulose, hydroxypropylmethylcellulose phthalate, noncrystalline cellulose, magnesium aluminum silicate, triethanolamine, polyvinyl alcohol, and polyvinylpyrrolidone (PVP).

The term “micronization” used herein means a process or method by which the size of a population of particles is reduced.

As used herein, the term “micron” or “μm” both are equivalent and refer to “micrometer” which is 1×10⁻⁶ meter.

As used herein, “crystalline particles” means any combination of single crystals, aggregates and agglomerates.

As used herein, “Particle Size Distribution (P.S.D)” means the cumulative volume size distribution of equivalent spherical diameters as determined by laser diffraction in Malvern Master Sizer 2000 equipment or its equivalent.

The important characteristics of the PSD are the (D₉₀), which is the size, in microns, below which 90% of the particles by volume are found, and the (D₅₀), which is the size, in microns, below which 50% of the particles by volume are found. Thus, a D₉₀ or d(0.9) of less than 300 microns means that 90 volume-percent of the particles in a composition have a diameter less than 300 microns.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. Solid state form of a salt of (±)-3-[2-[4-(6-fluoro-1,2-benzisoxazol-3-yl)-1-piperidinyl]ethyl]-6,7,8,9-tetrahydro-9-hydroxy-2-methyl-4H-pyrido[1,2-a]pyrimidin-4-one (paliperidone salt), wherein the salt of paliperidone is an L-(+)-tartrate salt, a tosylate salt, a maleate salt, an oxalate salt, an acetate salt or a malate salt.

2. The solid paliperidone salt of claim 1, which is in a crystalline form or an amorphous form, wherein the solid state form is anhydrous and/or solvent-free form, or a hydrate and/or a solvate form.

3. The solid paliperidone salt of claim 1, having the following characteristics, wherein:
a) the solid state form of paliperidone L-(+)-tartrate salt is characterized by one or more of the following properties:
i) a powder X-ray diffraction pattern substantially in accordance with FIG. 1;
ii) a powder X-ray diffraction pattern having peaks at about 10.32, 11.88, 16.35, 17.93, 21.10 and 21.48±0.2 degrees 2-theta;
iii) a powder X-ray diffraction pattern having additional peaks at about 11.43, 12.52, 12.88, 16.59, 18.15, 18.83, 20.61, 22.41, 23.93, 25.95, 26.45, 26.73, 28.25, 28.89, 29.13 and 33.56±0.2 degrees 2-theta; and
iv) a differential scanning calorimetric (DSC) thermogram substantially in accordance with FIG. 2;
b) the solid state form of paliperidone tosylate salt is characterized by one or more of the following properties:
i) a powder X-ray diffraction pattern substantially in accordance with FIG. 3;
ii) a powder X-ray diffraction pattern having peaks at about 7.08, 8.83, 14.28, 15.26, 17.09, 18.69 and 23.49±0.2 degrees 2-theta;
iii) a powder X-ray diffraction pattern having additional peaks at about 7.75, 11.24, 12.23, 13.59, 16.58, 17.75, 20.04, 21.24, 22.55, 24.29, 25.10 and 28.49±0.2 degrees 2-theta; and
iv) a differential scanning calorimetric (DSC) thermogram substantially in accordance with FIG. 4;
c) the solid state form of paliperidone maleate salt is characterized by one or more of the following properties:
i) a powder X-ray diffraction pattern substantially in accordance with FIG. 5;
ii) a powder X-ray diffraction pattern having peaks at about 9.25, 11.02, 16.21, 16.47, 18.50 and 22.38±0.2 degrees 2-theta;
iii) a powder X-ray diffraction pattern having additional peaks at about 6.82, 8.78, 11.67, 12.01, 14.98, 16.80, 19.38, 20.49, 20.85, 23.45, 24.92, 26.10, 27.07, 27.63, 28.09, 29.11 and 30.56±0.2 degrees 2-theta; and
iv) a differential scanning calorimetric (DSC) thermogram substantially in accordance with FIG. 6;
d) the solid state form of paliperidone oxalate salt is characterized by one or more of the following properties:
i) a powder X-ray diffraction pattern substantially in accordance with FIG. 7;
ii) a powder X-ray diffraction pattern having peaks at about 13.32, 22.53, 23.78 and 27.73±0.2 degrees 2-theta;
iii) a powder X-ray diffraction pattern having additional peaks at about 6.92, 11.60, 12.92, 15.32, 15.93, 16.60, 17.47, 19.22, 20.11, 24.74 and 27.18±0.2 degrees 2-theta; and
iv) a differential scanning calorimetric (DSC) thermogram substantially in accordance with FIG. 8;
e) the solid state form of paliperidone acetate salt is characterized by one or more of the following properties:
i) a powder X-ray diffraction pattern substantially in accordance with FIG. 11;
ii) a powder X-ray diffraction pattern having peaks at about 8.16, 10.28, 13.78, 20.68, 24.66 and 25.06±0.2 degrees 2-theta;
iii) a powder X-ray diffraction pattern having additional peaks at about 7.43, 13.12, 14.54, 14.94, 17.57, 18.63, 19.23, 20.04, 27.97, 30.95 and 31.22±0.2 degrees 2-theta; and
iv) a differential scanning calorimetric (DSC) thermogram substantially in accordance with FIG. 12; and
f) the solid state form of paliperidone malate salt is characterized by one or more of the following properties:
i) a powder X-ray diffraction pattern substantially in accordance with FIG. 13;
ii) a powder X-ray diffraction pattern having peaks at about 10.23, 11.86, 16.33, 17.82, 20.90, 21.40 and 26.46±0.2 degrees 2-theta;
iii) a powder X-ray diffraction pattern having additional peaks at about 9.35, 9.97, 11.40, 12.32, 12.73, 16.58, 18.76, 2062, 22.25, 23.83, 25.71, 27.73, 28.03, 28.25, 28.97 and 33.58±0.2 degrees 2-theta; and
iv) a differential scanning calorimetric (DSC) thermogram substantially in accordance with FIG. 14.

4. A process for the preparation of solid paliperidone salt of claim 1, comprising:
a) providing a first solution or a suspension of paliperidone free base in a first solvent;
b) combining the first solution or suspension with an acid to produce a second solution or suspension containing paliperidone acid addition salt, wherein the acid is selected from the group consisting of L-(+)-tartaric acid, p-toluenesulfonic acid, maleic acid, oxalic acid, acetic acid and malic acid; and
c) optionally, substantially removing the solvent from the second solution or suspension to obtain a residue followed by dissolving or suspending the residue in a second solvent to produce a third solution or suspension;
d) isolating and/or recovering the solid state form of paliperidone salt either from the second solution or suspension obtained in step-(b) or from the third solution or suspension obtained in step-(c);
wherein the first and second solvents used in steps-(a) and (c) are, each independently, selected from the group consisting of water, an alcohol, a ketone, a chlorinated hydrocarbon, a hydrocarbon, an ester, a nitrile, an ether, a polar aprotic solvent, and mixtures thereof.

5. The process of claim 4, wherein the first and second solvents used in steps-(a) and (c) are, each independently, selected from the group consisting of water, methanol, ethanol, n-propanol, isopropyl alcohol, isobutanol, n-butanol, tert-butanol, amyl alcohol, isoamyl alcohol, hexanol, acetone, methyl ethyl ketone, methyl isobutyl ketone, methyl tert-butyl ketone, acetonitrile, ethyl acetate, methyl acetate, isopropyl acetate, tert-butyl methyl acetate, ethyl formate, methylene chloride, ethylene dichloride, chloroform, n-pentane, n-hexane, n-heptane, cyclohexane, toluene, xylene, tetrahydrofuran, dioxane, diethyl ether, diisopropyl ether, monoglyme, diglyme, N,N-dimethylformamide, N,N-dimethylacetamide, dimethylsulfoxide, and mixtures thereof.

6. The process of claim 5, wherein the first solvent is selected from the group consisting of water, methanol, ethanol, isopropyl alcohol, acetone, n-hexane, n-heptane, cyclohexane, and mixtures thereof; and wherein the second solvent is selected from the group consisting of tetrahydrofuran, dioxane, diethyl ether, diisopropyl ether, monoglyme, diglyme, and mixtures thereof.

7. The process of claim 4, wherein the first solution in step-(a) is prepared by dissolving paliperidone free base in the first solvent at a temperature of about 0° C. to the reflux temperature of the solvent; wherein the suspension in step-(a) is provided by suspending paliperidone free base in the first solvent while stirring at a temperature of about 0° C. to the reflux temperature of the solvent used; and wherein the first solution or suspension obtained in step-(a) is optionally stirred at a temperature of about 25° C. to the reflux temperature of the solvent used for at least 15 minutes.

8. The process of claim 4, wherein the combining in step-(b) is accomplished by adding the first solution or suspension to the acid or by adding the acid to the first solution or suspension, at a temperature of about 0° C. to the reflux temperature of the solvent; wherein the acid in step-(b) is used directly or in the form of a solution containing the acid and a solvent, wherein the solvent is selected from the group consisting of water, methanol, ethanol, n-propanol, isopropyl alcohol, isobutanol, n-butanol, tert-butanol, amyl alcohol, isoamyl alcohol, hexanol, acetone, methyl ethyl ketone, methyl isobutyl ketone, methyl tert-butyl ketone, acetonitrile, ethyl acetate, methyl acetate, isopropyl acetate, tert-butyl methyl acetate, ethyl formate, methylene chloride, ethylene dichloride, chloroform, n-pentane, n-hexane, n-heptane, cyclohexane, toluene, xylene, tetrahydrofuran, dioxane, diethyl ether, diisopropyl ether, monoglyme, diglyme, N,N-dimethylformamide, N,N-dimethylacetamide, dimethylsulfoxide, and mixtures thereof.

9. The process of claim 4, wherein the removal of solvent in step-(c) is accomplished by substantially complete evaporation of the solvent, concentrating the solution or distillation of solvent under inert atmosphere, or a combination thereof; wherein the residue containing paliperidone acid addition salt obtained in step-(c) is dissolved or suspended in the second solvent a temperature of about 0° C. to the reflux temperature of the solvent; wherein the isolation of pure solid state form of paliperidone salt in step-(d) is carried out by cooling, seeding, partial or substantial removal of the solvent from the solution or suspension, by adding an anti-solvent to the solution, or a combination thereof; wherein the recovering in step-(d) is carried out by filtration, filtration under vacuum, decantation, centrifugation, filtration employing a filtration media of a silica gel or celite, or a combination thereof; and wherein the substantially pure solid state form of paliperidone salt obtained in step-(d) is further dried under vacuum or at atmospheric pressure, at a temperature of about 35° C. to about 90° C.

10. The process of claim 9, wherein the isolation in step-(d) is carried out by cooling the solution under stirring at a temperature of about 0° C. to about 30° C. for about 30 minutes to about 20 hours.

11. A process for preparing highly pure paliperidone free base using the solid state form of paliperidone salt of claim 1, comprising:
a) contacting solid state form of a paliperidone salt with a base in a first solvent to provide a reaction mass containing paliperidone free base, wherein the salt of paliperidone is an L-(+)-tartrate salt, a tosylate salt, a maleate salt, an oxalate salt, an acetate salt or a malate salt; and
b) optionally, recovering the paliperidone free base from the reaction mass obtained in step-(a) and followed by extracting, suspending or dissolving the paliperidone free base in a second solvent;
c) isolating and/or recovering the pure paliperidone free base either from the reaction mass obtained in step-(a) or from the solution or suspension obtained in step-(b);
wherein the first and second solvents used in steps-(a) and (b) are, each independently, selected from the group consisting of water, an alcohol, a ketone, a chlorinated hydrocarbon, a hydrocarbon, an ester, a nitrile, an ether, a polar aprotic solvent, an organosulfur solvent, and mixtures thereof.

12. The process of claim 11, wherein the first and second solvents used in steps-(a) and (b) are, each independently, selected from the group consisting of selected from the group consisting of water, methanol, ethanol, n-propanol, isopropyl alcohol, isobutanol, n-butanol, tert-butanol, amyl alcohol, isoamyl alcohol, hexanol, acetone, methyl ethyl ketone, methyl isobutyl ketone, methyl tert-butyl ketone, acetonitrile, ethyl acetate, methyl acetate, isopropyl acetate, tert-butyl methyl acetate, ethyl formate, methylene chloride, ethylene dichloride, chloroform, n-pentane, n-hexane, n-heptane, cyclohexane, toluene, xylene, tetrahydrofuran, dioxane, diethyl ether, diisopropyl ether, monoglyme, diglyme, N,N-dimethylformamide, N,N-dimethylacetamide, dimethylsulfoxide, sulfolane, and mixtures thereof; and wherein the base used in step-(a) is an organic or inorganic base.

13. The process of claim 12, wherein the first solvent is selected from the group consisting of water, methanol, ethanol, isopropyl alcohol, acetone, sulfolane, and mixtures thereof; wherein the second solvent is selected from the group consisting of methanol, ethanol, n-propanol, isopropyl alcohol, acetone, sulfolane, and mixtures thereof; and wherein the base is selected from the group consisting of triethyl amine, dimethyl amine, tert-butyl amine, aqueous ammonia, sodium hydroxide, calcium hydroxide, magnesium hydroxide, potassium hydroxide, lithium hydroxide, sodium carbonate, potassium carbonate, lithium carbonate, sodium bicarbonate and potassium bicarbonate.

14. The process of claim 11, wherein the contacting in step-(a) is carried out under stirring at a temperature of about 0° C. to about the reflux temperature of the solvent used for about 15 minutes to about 15 hours; wherein the isolation of pure paliperidone free base in step-(c) is carried out by forcible crystallization, spontaneous crystallization, substantial removal of the solvent from the solution or suspension, or a combination thereof; and wherein the recovering in steps-(b) and (c) is carried out by filtration, filtration under vacuum, decantation, centrifugation, filtration employing a filtration media of a silica gel or celite, or a combination thereof.

15. A crystalline Form II of paliperidone fumarate characterized by one or more of the following properties:
i) a powder X-ray diffraction pattern substantially in accordance with FIG. 9;
ii) a powder X-ray diffraction pattern having peaks at about 10.65, 14.16, 15.79, 19.87, 20.17, 21.45 and 25.70±0.2 degrees 2-theta;
iii) a powder X-ray diffraction pattern having additional peaks at about 9.93, 11.43, 17.43, 18.99, 20.58 and 24.09±0.2 degrees 2-theta;
iv) a powder X-ray diffraction pattern having no peaks at about 7.60, 8.21, 13.81, 15.23, 26.98 and 29.11±0.2 degrees 2-theta; and
v) a differential scanning calorimetric (DSC) thermogram substantially in accordance with FIG. 10.

16. A process for the preparation of paliperidone fumarate crystalline Form II of claim 15, comprising:
a) providing a suspension of paliperidone free base in ethanol;
b) combining the suspension with fumaric acid to produce a reaction mass containing paliperidone fumarate; and
c) isolating and/or recovering the crystalline Form II of paliperidone fumarate from the reaction mass obtained in step-(b).

17. The solid paliperidone salt of claim 1, further comprising one or more pharmaceutically acceptable excipients to form a pharmaceutical composition.

18. The pharmaceutical composition of claim 17, wherein the pharmaceutical composition is a solid dosage form, an oral suspension, a liquid, a powder, an elixir, an aerosol, syrups or an injectable solution.

19. The pharmaceutical composition of claim 17, wherein the solid state form of paliperidone salt has a D₉₀ particle size of less than or equal to about 500 microns.

20. The pharmaceutical composition of claim 19, wherein the D₉₀ particle size is about 1 micron to about 300 microns, or about 10 microns to about 150 microns.

21. (canceled)
